(19) United States
(12) Patent Application Publication
(10) Pub. No.: US 2008/0228066 A1
(43) Pub. Date: Sep. 18, 2008

(54) METHODS AND SYSTEMS FOR LOCATING A FEEDING TUBE INSIDE OF A PATIENT

(76) Inventor: Kathryn A. McKenzie Waitzman, Riverwoods, IL (US)

Correspondence Address: MCANDREWS HELD & MALLOY, LTD, 500 WEST MADISON STREET, SUITE 3400, CHICAGO, IL, 60661, US

(21) Appl. No.: 12/048,500

(22) Filed: Mar. 14, 2008

Related U.S. Application Data
(60) Provisional application No. 60/906,981, filed on Mar. 14, 2007.

(51) Int. Cl. A61B 5/06

(52) U.S. Cl. 600/424

(57) ABSTRACT

Systems and methods are provided for detecting the position of a feeding tube in a patient's body. A feeding tube having a light source sensor is inserted into a patient. A light is generated over the body of a patient. When the light source sensor detects the presence of the light, a signal is generated. In certain embodiments, the signal may be based upon the intensity of the light perceived at the light source sensor. Certain embodiments provide systems and methods comprising a feeding tube having a proximity sensor target. A detector having a proximity sensor is moved externally about a patient's body such that, when the sensor detects the proximity sensor target of the feeding tube, the proximity sensor produces an indication signal.

### CROSS-REFERENCE TO RELATED APPLICATIONS

This non-provisional application claims priority to U.S. provisional application No. 60/906,981 titled “Feeding Tube Detector” filed Mar. 14, 2007.

### FIELD OF THE INVENTION

Embodiments of the presently described technology generally relate to techniques to confirm the location of a medical device in a medical patient's body. More particularly, embodiments of the present technology relate to methods and systems for locating and confirming the end of a particular feeding tube that has been inserted into a patient without exposing the patient to radiation.

### BACKGROUND OF THE INVENTION

Feeding tube intubation is a process involving placement of a soft plastic tube into a patient's stomach or jejunum, otherwise referred to as the small intestine. Typically, the gastric or intestinal feeding tube is inserted through a patient's nose or mouth and travels past the pharynx, down the esophagus and into a patient's stomach or beyond to the small intestine. Intubation is a common medical practice that may assist in the treatment and diagnosis of patients. For example, the intubation of a gastric feeding tube can aid a patient in recovery from surgery or trauma by administering life sustaining nutrition or medications where necessary. Patients who need gastric or intestinal feeding tubes include but are not exclusive to pre-mature neonates, comatose patients, patients requiring mechanical ventilation, chronically ill children, patients requiring face or neck surgeries, cancer patients, and/or post-op surgical nutrition. The feeding tubes are considered temporary, non-surgical, and intended to remain in use for short-term or long-term therapies until a trained physician deems a change medically necessary.

Gastric feeding tube placement is routinely practiced in both medical facilities and in the treatment of in-home-care patients. Intestinal feeding tube placement frequently requires the use of more specialized placement techniques and the placement position is more difficult to confirm in placement. As such, intestinal feeding tube placement is predominantly practiced only in medical facilities.

Feeding tubes are routinely placed in patients using a blind technique, with the operator not knowing the true location of the end of the tube after placement. Accordingly, the end of the feeding tube is commonly misplaced inside of the patient, which may lead to serious problems. For example, where a feeding tube intended for placement in the stomach is not placed deep enough, fluids administered through the feeding tube may seep into the lungs causing problems for the patient. Alternatively, where such a feeding tube is placed too deep, the fluids may be absorbed directly into the intestine, which may not have the appropriate enzymes for processing the fluids, which may also lead to problems. Complications that may result from the improper administration of fluids through an improperly placed feeding tube may include, but are not limited to, pneumothorax, perforated bowel, pneumonia, intestinal distention, aspiration, peritonitis, or placement of the tube into the brain, for example. See, Ellet, Maahs, and Forsee, Prevalence of Feeding Tube Placement Errors and Associated Risk Factors in children, American Journal Maternal Child Nursing, 23:234-39, published 1998; Ellet, What is Known About Methods Of Correctly Placing Gastric Tubes in Adults and Children, Gastroenterology Nursing, 27 (6):253-59, published 2004; Ellet, What is the Prevalence of Feeding Tube Placement Errors and What are the Associated Risk Factors?, The Online Journal of Knowledge Synthesis for Nursing, 4, document 5, published 1997.

The misplacement of feeding tubes in patients happens frequently when blind insertion techniques are used. Research has suggested that blind placement methods of feeding tubes may have a mal-position rate in pediatric and adult patients of up to 40%. See Metheny and Tiller Assessing Placement of Feeding Tubes, American Journal of Nursing, 101:36-41, published 2001; Methney and Meert, Monitoring Feeding Tube Placement, Nutrition in Clinical Practice, Vol. 19, no. 5, pp. 487-95, published 2004; Huffman, Karczk, O'Brien, Pieper and Bayne, Methods to Confirm Feeding Tube Placement: Application of Research in Practice, Pediatric Nursing, 30:10-13, published 2004; Westhaus, Methods to Test Feeding Tube Placement in Children, The American Journal of Maternal/Child Nursing, 29:282-87, published 2004; Ellet, How Accurate is Entreal Tube Placement in Children?, MNRS Connection, 14 (1), 14, published 1998. Accordingly, it is often necessary to confirm the location of the feeding tube prior to the administration of any medication or nutrition to avoid problems caused by feeding tube misplacement.

Conventional methods for locating the position of a feeding tube or tubes inside a patient include the use of air insufflation, gastric pH detection methods, gastric enzyme detectors and CO₂ detectors. There are problems, however, with the accuracy and reliability of these methods. See Gharpure, Meert, Sarnaik and Metheny, Indicators of Postpyloric Feeding Tube Placement in Children, Critical Care Medicine, 28:2962-66, published 2000; Metheny, Stewart, Smith, Yan, Diebold and Clouse, pH and Concentration of Bilirubin in Feeding Tube Aspirates as Predictors of Tube Placement, Nursing Research 48, 189-97, published 1999; Araujo-Preza, Melhado, Gutierrez, Maniatis and Castellano, Use of Capnometry to Verify Feeding Tube Placement, Critical Care Medicine, 30:2255-2259, published 2002. For example, air insufflation techiques require a user to confirm the location of a tube by listening for a sound of air passing through a feeding tube inside the patient using a stethoscope. Internal noises may lead to a false confirmation of proper placement, for example. Furthermore, feedings and medications may affect the levels of pH, enzyme and CO₂ in a patient, thereby affecting the ability of gastric pH, gastric enzymes, and CO₂ detectors to produce accurate and reliable results.

Moreover, conventional methods typically require the implementation of equipment that is only available in a hospital or clinical setting and are thus unavailable for use with in-home-care patients. Presently, only air insufflation, the least accurate of the methods, is available to confirm proper placement of feeding tubes for in-home-care patients.

In June 2005, the American Association of Critical-Care Nurses (AACN) issued a practice alert. The alert recommended using an X-ray to visualize a new, blindly inserted gastric tube to ensure that the tube has been properly placed and is in the desired position of the stomach or small intestine before initiating the administration of formula or medications via the tube. See American Association of Critical Care Nurses, Practice Alert—Verification of Feeding Tube Placement, May 2005. Though more accurate than the conventional methods described above, the use of such techniques typically requires at least 5 X-ray scans to confirm the location of the tube, for each time an intestinal feeding tube is inserted blindly at a patients hospital bedside. It is not uncommon for children and neurologically compromised patients to personally remove/extubate the OG or NG tubes more than one time daily which would require additional X-rays for each new tube placement. Such persistent exposure to X-rays throughout a patient's treatment gives rise to serious concern, as the high levels of radiation can have harmful effects on the patient. This concern is especially great where the patient is a child. An additional disadvantage for using X-ray techniques to confirm feeding tube placement is that the equipment necessary to perform the techniques is typically only available in hospital environments and thus of no help to in-home-care patients.

Recently, the use of electromagnetic tube placement devices has provided a means to increase the accuracy of feeding tube placement without the need for X-ray exposure to patients. An example of such a device is the CORTRAK™ system produced by Cardinal Health. (A description of the product is available on the Cardinal Health website, at www.viasyshealthcare.com/prod_serv/prodDetail.aspx?config=ps_prodDtl&prodID=276, as of Mar. 11, 2008). The electromagnetic systems involve the placement of an electromagnetic transmitter inside of the feeding tube. As the tube is inserted into the patient, an electromagnetic tracking device tracks the position of the feeding tube, and displays the location on a display unit. Accordingly, operators can respond immediately where a tube placement does not follow the expected path. Because these techniques are only available in medical facilities, they are not helpful when needed for in-home-care.

Once the feeding tube has been inserted into the patient using the aforementioned electromagnetic tracking techniques, the transmitter device must be removed before feedings or medications can be administered through the tube. After the transmitter has been removed, however, it may not be reinserted without the removal of the feeding tube. Accordingly, once the transmitter has been removed, the position of a feeding tube inside the patient may not be checked. This shortcoming of the electromagnetic system is significant, as patient movement, periodic adjustment of the equipment, peristalsis and other internal functions all contribute to constant shifting and relocation of the feeding tube. Thus, it is necessary to periodically confirm the position of a feeding tube, even after it has been inserted. Without the transmitter located in the tube, the electromagnetic tracking techniques cannot confirm the position after insertion without the use of X-rays.

Thus, the concerns with the present feeding tube placement practices and techniques include several problems relating to accuracy, safety and ease of use for in-home-care patients. Thus a need exists for a method and/or system for detecting, and periodically re-checking, the location of a placed feeding tube in a patient's stomach or small intestine that has the accuracy of X-ray detection without the radiation exposure.

### SUMMARY OF THE INVENTION

Certain embodiments of the presently described technology provide a system for detecting the location of a feeding tube inside of a patient's body. The system provides a feeding tube embedded with a light source sensor. In certain embodiments, the light source sensor may be a passive infrared sensor or a fiber-optic filament or filaments, for example. The light source sensor is connected to a receiver. The system also provides a light source that generates non-radiographic light. For example, the light source may generate infrared light. The light source generates light over the body of a patient in which a feeding tube having a light source sensor has been inserted. When the light source sensor detects light from the non-radiographic light source, the receiver generates a signal based on the detection of the light. In certain embodiments, the receiver generates a signal based on the intensity of the light detected. The signal may be audible or visual, and the signal may change based on the intensity of light detected by the sensor. For example, the signal may be a tone that increases in volume or pitch as the intensity of light perceived by the sensor increases.

Certain embodiments of the presently described technology provide a method for locating the end of a feeding tube in a patient's body. The method comprises inserting a feeding tube having a light source sensor located at the distal end of the tube into a patient. In certain embodiments, the sensor is connected to a receiver, and the feeding tube is inserted into the patient. Next, a non-radiographic light is generated over the patient. The sensor detects the light, and the receiver generates a signal based on the detection of light. In certain embodiments, the receiver generates a signal based on the intensity of light detected at the light source sensor. In certain embodiments, the non-radiographic light source is moved externally over the patient to the point where the indicator signal generated by the receiver exceeds a predetermined intensity threshold, where the predetermined threshold is sufficient to confirm the presence of the feeding tube.

Certain embodiments provide a system for locating the end of a feeding tube in a patient's body using a proximity sensor and a proximity sensor target. The system comprises a feeding tube having a proximity sensor target. The sensor target may be, for example, a metal band or metal object of some sort. The system also provides a detector having a proximity sensor. For example, the proximity sensor may be a metal detector. The detector may move externally over a patient's body such that, when the sensor detects the proximity sensor target of the feeding tube, the proximity sensor produces an indication signal.

### BRIEF DESCRIPTION OF SEVERAL VIEWS OF THE DRAWINGS

FIG. 1 illustrates an isometric view of a feeding tube according to an embodiment of the present technology.

FIG. 2 illustrates a top view of a detector according to an embodiment of the present technology.

FIG. 3 illustrates a side view of the detector of FIG. 2.

FIG. 4 illustrates a bottom view of the detector of FIG. 2.

FIG. 5 illustrates an isometric view of a feeding tube detection system according to an embodiment of the present technology.

FIG. 6 illustrates a top view of the system of FIG. 5.

FIG. 7 illustrates a cross section of a feeding tube of the system of FIG. 5.

FIG. 8 illustrates a schematic diagram of the system of FIG. 5.

Before the embodiments of the technology are explained in detail, it is to be understood that the technology is not limited in its application to the details of construction and the arrangement of the components set forth in the following description or illustrated in the drawings. The technology is capable of other embodiments and of being practiced or being carried out in various ways. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof.

### DETAILED DESCRIPTION OF THE INVENTION

Proximity sensors are sensors that are able to detect the presence of nearby objects without physical contact. A proximity sensor emits an electromagnetic or electrostatic field, or a beam of electromagnetic radiation such as infrared, for example. The proximity sensor looks for changes in the field or return signal. The object being sensed is often referred to as a proximity sensor target. Different proximity sensor targets demand different sensors. For example, a capacitive or photoelectric sensor might be suitable for a plastic target, and an inductive proximity sensor may be suitable for a metal target. Proximity sensors have a maximal distance at which they can be detected. The range in which the sensor can be detected is called the “nominal range.” Certain sensors may have the ability to adjust the nominal range, or provide a way to report graduated detection distance. Proximity sensors have a high reliability and a long functional life because of the absence of mechanical parts and the lack of physical contact between the sensor and the sensed object.

A capacitive proximity sensor is a variety of proximity sensor that detects the location of an object between two capacitor plates. When the sensed object moves within the nominal range of the sensor, the dielectric constant between two plates changes, and the position of the object can thus be located. One type of capacitive proximity sensor is a metal detector. One embodiment of the present technology employs the use of a metal detector capacitive proximity sensor to detect the location of a feeding tube inside of a patient.

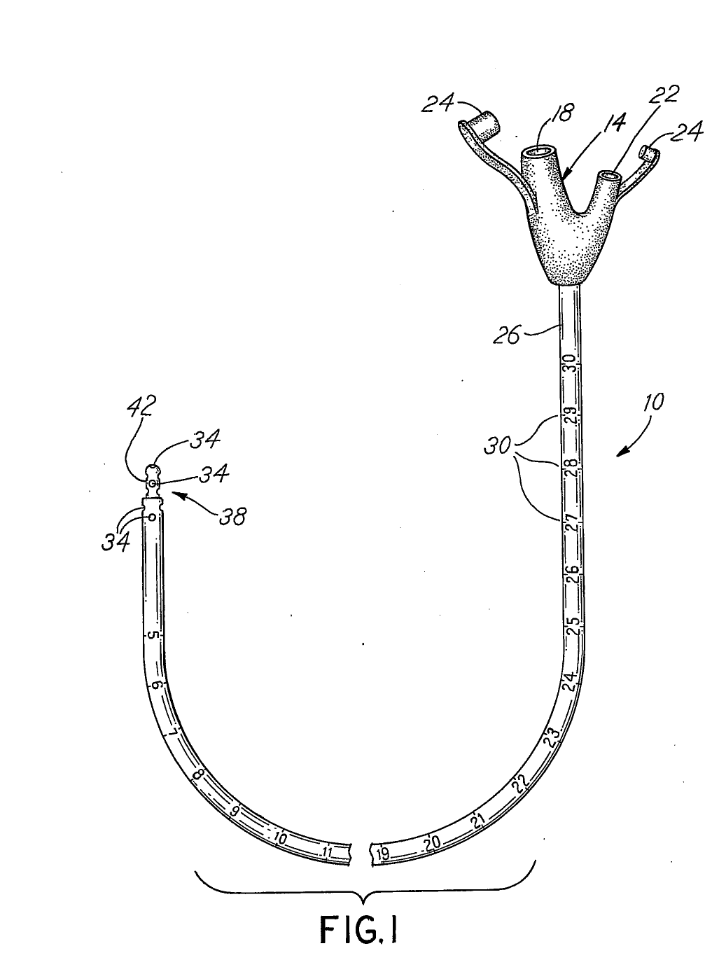

FIG. 1 illustrates an isometric view of a feeding tube system 10 according to an embodiment of the present technology. The feeding tube system 10 has a “Y” port 14 with dual administration ports 18 and 22. The dual ports 18 and 22 may be used simultaneously to administer feedings and medications. For example, the port 18 may be used to receive gastric feeding and the port 22 may be used for medication, flushing, or as a racking port, or vice versa. The “Y” port 14 includes caps 24 connected thereto that may be used to close the administration ports 18 and 22. The “Y” port 14 is connected to a main tube 26, or a feeding tube.

For identification purposes, the tube 26 may also be referred to as a “feeding tube,” an “oral gastric tube (or OG-tube),” a “nasogastric tube (or NG-tube),” or an “intestinal tube,” depending on the location of placement of the tube inside of a patient for treatment. The tube 26 may be comprised of a variety of materials, for example, polyurethane. In certain embodiments, the tube 26 may comprise location or measurement markings 30 along the length of the tube 26 from the “Y” port 14 to a distal end 38. The markings 30 may be used as a guide to determine the location of, or the amount of the tube exposed. For example, the markings may be numbered and consistently spaced apart to measure length in, by way of example only, centimeters. In certain embodiments, an operator may insert the feeding tube 26 to a predetermined depth using the markings 30 as a guide before attempting to confirm location of the end of the tube 26.

The tube 26 may include a radiographic pigment indicator line. The radiographic pigment indicator line allows the tube 26 to appear on an X-ray, should confirmation by X-ray be necessary. Holes 34 may be situated at the distal end 38 of the tube 26 for fluid administration into the patient. In certain embodiments, the tube 26 may also comprise a proximity sensor target 42 impregnated into the distal end 38 of the tube 26. In certain embodiments, the proximity sensor target 42 may be a metal band or a metal object of some kind, for example. In operation, where the target 42 is a metal, it is preferred that it be comprised of a non-ferrous metal that is not otherwise located in the patient's body. For example, where a patient has an implant made of titanium, it is preferred that the metal band 42 be comprised of a non-ferrous metal that is not titanium. By way of example only, the metal sensor may be comprised of zinc or silver.

In certain embodiments, the tube 26 may be for single use and be non-sterile. In other embodiments, however, the tube 26 may be reusable. The tube 26 may range in size from 3.5 French (or approximately having a circumference of 3.5 millimeters) to 12 French (approximately 12 millimeters in circumference), for example, and may vary in length. For example, the tube may be, by way of example only, 36 to 42 inches in length.

In operation, the distal end 38 of the feeding tube 10 may be inserted into the stomach or small intestine of a patient by inserting the tube 26 through a patient's nose or mouth, and down the patient's esophagus such that the distal end 38 locates in the patient's stomach, or through the stomach and into the small intestine. Once situated, food and medication may then be fed into the ports 18 and 22, through the tube 26, and into the patient through the holes 34 at the distal end 38.

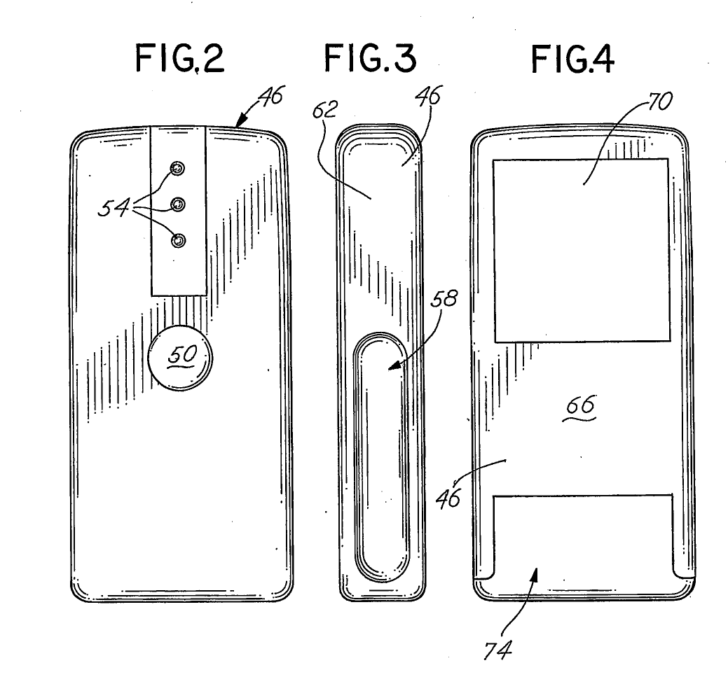

FIG. 2 illustrates a top view of a sensor or detector 46 according to an embodiment of the present technology. The detector 46 is used to determine the position of the proximity sensor target 42 at the distal end 38 of the feeding tube 10. The detector 46 is generally rectangular in shape and sized to be hand-held. By way of example only, the detector 46 may have a housing made of hard plastic.

In certain embodiments, the detector 46 internally carries a proximity sensor. In certain embodiments where the proximity sensor target is metal, such as is described above, the proximity sensor may be a metal detector. Externally, the detector includes an on/off button or switch 50 and indicator lights 54. In certain embodiments the detector may include a speaker instead of, or in addition to, the indicator lights 54. In certain embodiments, the detector 46 is designed to be operable with either a right or a left hand. For example, a user holding the detector 46 with a left hand only may be able to operate the on/off button, or any other functions, as would a user holding the detector 46 with the right hand only. The metal detector or proximity sensor does not generate X-rays.

In certain embodiments, the proximity sensor may be capable of scanning for sensor targets (e.g., metal where the proximity sensor is a metal detector) at least at two various depths. For example, the proximity sensor may have a regular depth of scanning and a deeper depth of scanning for obese patients. The detector 46 may have buttons or switches that allow the operator to set the depth of the scan to regular or deep. Alternatively, the detector 46 may be used to scan at any number of different depths.

FIG. 3 illustrates a side view of the detector 46 of FIG. 2. The detector 46 includes finger grips 58 along sidewalls 62 thereof. The finger grips may provide for easier gripping of the detector 46 by an operator and may be made of rubber or plastic.

FIG. 4 illustrates a bottom view of the detector 46 of FIG. 2. In certain embodiments, the detector 46 has a back side 66 that includes a section 70 for displaying information, for example, use, cleaning and warning instructions. In certain embodiments, the back side 66 also includes a compartment 74 that may be opened and closed to receive a battery. By way of example only, the detector 46 may operate on a 9-volt battery. In certain embodiments, the detector 46 may operate with a lithium ion battery. In certain embodiments, the detector 46 may operate on alternating current or an alternative power source.

Returning to FIG. 2, in operation, an operator presses the button 50 to turn on the detector 46. The operator then moves the backside 66 (FIG. 4) of the detector 46 to the area of the patient where the feeding tube 26 is assumed to be situated. For example, where the feeding tube is intended to be located in the patient's stomach, the operator moves the backside 66 of the detector 46 externally over the stomach area of a patient. The indicator lights 54 may be of a certain color or indication signal when the detector 46 does not detect the proximity sensor target 42 of the feeding tube 10. For example, the indicator lights 54 may be red when the metal detector 46 does not detect the sensor target 42 of the tube 10 in the patient's stomach. When the proximity sensor of the detector 46 detects the sensor target 42 of the feeding tube 10, the lights turn to a different color or indicator signal to indicate that the detector has detected the position of the sensor target 42, and thus the distal end 38 of the feeding tube 10. For example, the indicator lights 54 may turn green to indicate that the sensor target 42 is detected. In another embodiment, the indicator lights 54 may be unilluminated when the detector 46 does not detect the target 42, and illuminated when the detector 46 detects the target 42. In an alternative embodiment, the detector 46 may emit an audible sound to confirm detection in addition to, or instead of, using the indicator lights 54. When the detector 46 has indicated that it has detected the sensor target 42, the operator will be able to determine whether the distal end 38 of the feeding tube is correctly positioned in the stomach.

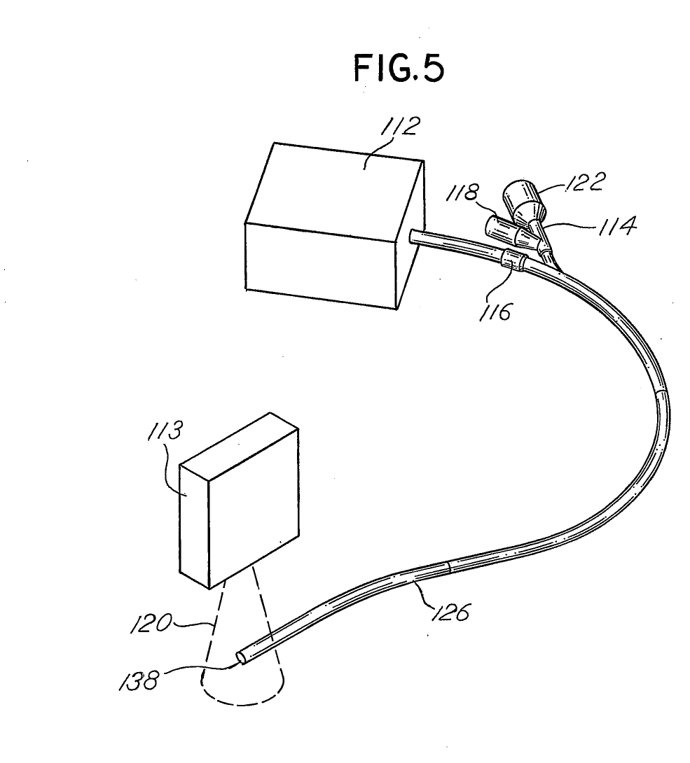

The embodiments depicted in FIGS. 1-4 and described supra involve use of a sensor target 42, such as a metal band, and a proximity sensor, such as a metal detector, as a way of detecting the position of a feeding tube inside of a patient. Other embodiments of the present technology provide alternative techniques for locating the position of a feeding tube inside of a patient without exposing the patient to radiation. For example, certain embodiments provide for a sensor on the feeding tube that detects the presence of a source external to the body. For example, FIGS. 5-8 depict embodiments of the present technology that use a light source sensor on the feeding tube and a non-radiographic light source to determine the location of the tube.

A passive infrared sensor (PIR sensor) is an electronic device that measures infrared light radiating from objects within the field of view. PIR sensors are often used in the construction of motion detectors. All objects emit an energy called “black body radiation.” This black body energy is invisible to the human eye, but can be detected by electronic devices. The term “passive” means that the sensor does not emit energy; instead, the sensor merely receives the energy. For example, a PIR sensor detects motion when one infrared source having one temperature, such as a human, passes in front of an infrared source having another temperature, such as a wall. The PIR sensor detects the change in energy between the sensor and the wall and transmits a signal that an object has been detected. Certain embodiments of the present technology employ related systems and methods to detect the presence of a feeding tube inside of a patient.

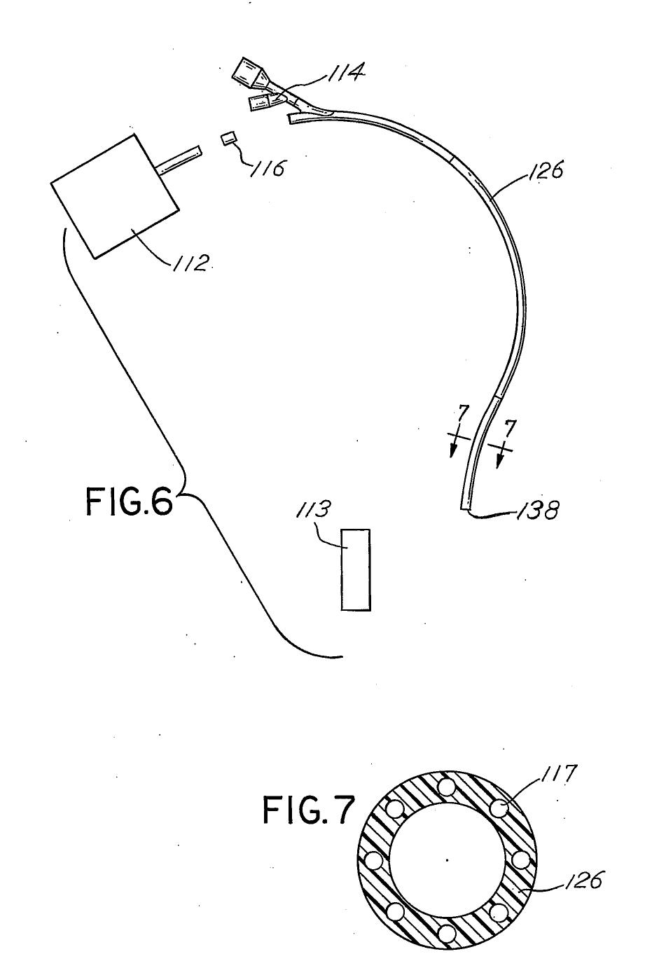

FIGS. 5 and 6 illustrate an isometric and blown up view of a feeding tube system 110, respectively, according to an embodiment of the present technology. The feeding tube 110 has a “Y” port 114 to administer feedings, medications or other treatments. The “Y” port 114 may comprise dual ports 118 and 122 such that multiple feedings, medications or other treatments may be administered simultaneously. For example, the port 118 may be used to receive gastric feeding and the port 122 may be used for medication, flushing, or as a racking port, or vice versa. The “Y” port 114 is connected to a main tube 126. The main tube 126 administers feedings, medications or other treatments to a patient internally, for example, through the stomach or the small intestine. The tube 126 may also be referred to as a “feeding tube,” an “oral gastric tube (or OG-tube),” a “nasogastric tube (or NG-tube),” or an “intestinal tube,” as it refers to the location of the tube when inserted into the patient. The tube 126 comprises a distal end 138, which may comprise a hole or holes for the administration of fluid into the patient. The feeding tube 126 comprises a light source sensor 117, which is connected to a receiver 112 via a joint 116. The joint 116 provides for removable connection of the tube 126 to the receiver 112. For example, when not in operation, the tube 126 may be disconnected to the receiver 112. In certain embodiments, the light source sensor 117 may be wirelessly connected to the receiver.

A cross section of the tube 126 is depicted in FIG. 7. The feeding tube 126 may be comprised of a variety of flexible materials suitable for insertion into a patient. A material such as medical grade polyurethane is, by way of example only, a suitable material type. In certain embodiments the feeding tube may comprise a radiographic pigment for X-ray detection. For example, the tube 126 may have a radiographic pigment stripe that will appear on an X-ray, indicating the position of the tube in the body. In certain embodiments the feeding tube 126 may comprise a series of markings, similar to the markings 30 of the tube 26 in FIG. 1. The markings may be used as a guide to determine the approximate location of the feeding tube inside the patient by indicating the amount of tube exposed. For example, the markings may be numbered and consistently spaced apart to measure length in, by way of example only, centimeters. Thus, a practitioner may insert the tube 126 to an approximately appropriate depth before attempting to locate the tube in the patient.

A light source sensor 117 runs along the outer wall of the tube 126 as shown in FIG. 7. The light source sensor may be, for example, a passive infrared sensor or a fiber-optic filament. In certain embodiments the light source sensor 117 is a plurality of fiber-optic filaments, such as is depicted in FIG. 7. The end of the light source sensor 117 is situated at the distal end 138 of the feeding tube 126. In certain embodiments, a feeding tube 126 may have more than one light source sensor; however, certain embodiments will employ only one light source sensor.

A light source 113 shines a non-radiographic light 120 through the body as shown in FIG. 5. In certain embodiments, the light source is an infrared light source, and the light shone is infrared light. When the light 120 is detected by the end of the light source sensor 117, a detection signal is transmitted up the sensor 117 to the receiver 112. In certain embodiments, the light source sensor 117 may appreciate a range of light intensity as opposed to merely the presence of light, or lack thereof. For example, the light source sensor 117 may detect the faint presence of the light source 113 when the light is within a particular range of the sensor 117. As the light source 113 moves closer to the distal end 138 of the tube 126 the intensity of light at the sensor 117 increases, and as the source 113 moves away from the end 138 of the tube, the light intensity at the sensor 117 decreases. In certain embodiments, the light source sensor 117 appreciates this change, and the receiver 112 generates an indicator signal to indicate that the light source 113 is closer to or farther from the end of the light source sensor 117 accordingly.

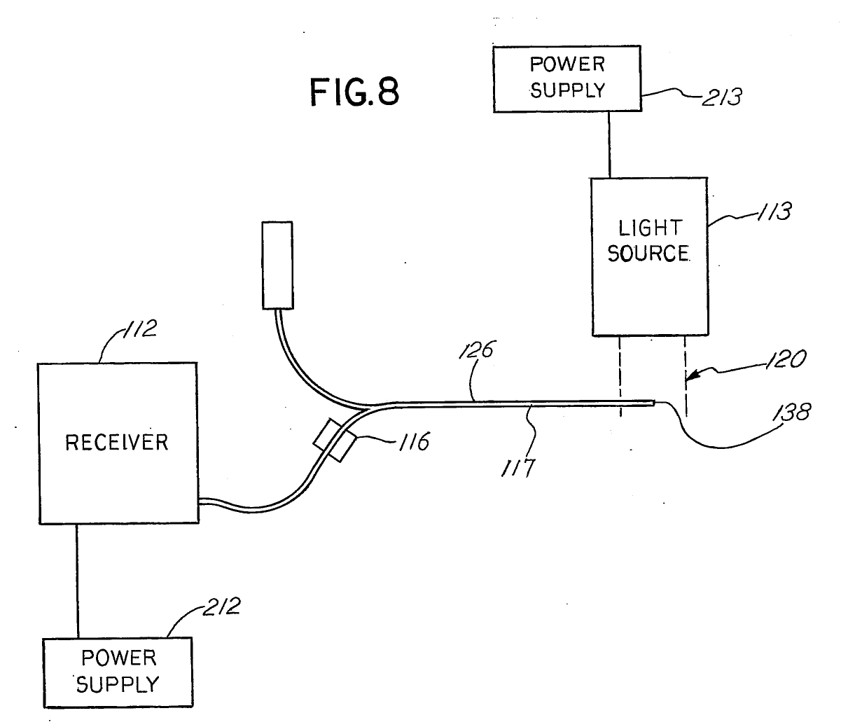

FIG. 8 depicts a schematic diagram of an embodiment employing the use of a light source sensor to locate a feeding tube inside of a patient in operation. A light source 113 is connected to a power supply 213. Similarly, receiver 112 is connected to a power supply 212. Power supplies 212 and 213 may be, for example, a 12-volt power supply, a battery, an alternating current source or an alternate power source. An operator moves the light source 113 externally above a patient in an area where the distal end 138 of the feeding tube 126 is approximately located. For example, when the feeding tube is intended to be placed inside of a patient's stomach, an operator may move the light source 113 externally, approximately above the patient's stomach.

When the light source is within the detected range of the light source sensor 117 embedded in the tube 126, the receiver 112, connected to the light source sensor 117, generates an indicator signal indicating that the light 120 has been perceived by the light source sensor 117. For example, the receiver may emit a sound indicating the light is detected, or may cause a sound to be emitted from another source such as a speaker. Alternatively, the receiver 112 may indicate perception of the light 120 through visual signals, such as an LED light or lights that blink, change color or otherwise indicate the light signal is perceived. The receiver 112 may also cause a display unit to produce a signal or a message that indicates that the light source sensor 117 detects the presence of light, or lack thereof.

In certain embodiments, as the light 120 draws nearer the actual location of the light source sensor 117, the intensity of the light detected increases. Accordingly, the intensity of the signal generated by the receiver 112 may increase. The receiver 112 may indicate a perceived increase in intensity by changing or modifying the signal produced. For example, the receiver 112 may produce a sound, such as a tone, that increases in pitch or volume as the intensity of light perceived increases. In certain embodiments the receiver 112 may produce a series of intermittent sounds wherein the amount of time between the sounds increases or decreases with an increase in intensity of light perceived by the light source sensor 117. For example, where the light source sensor 117 detects no light, the receiver 112 may produce a “chirp” once every two seconds, or not at all. As the light source sensor 117 detects an increase in the intensity of light, the receiver may reduce the time between the “chirps” produced, such that when the light intensity perceived by the light source sensor 117 is at a maximum, the receiver produces one “chirp” every 0.1 seconds, for example.

Alternatively or additionally, the receiver 112 may have a visual display to generate the signal. For example, in certain embodiments, the receiver 112 may have a blinking light that modifies the frequency of the blinking with a change in intensity. Additionally, the receiver may provide a series of LED lights, where the particular light, or the number of lights illuminated, indicates the intensity of the signal. For example, the receiver may have a bar of ten lights on the receiver, each light corresponding to a particular intensity level. When the intensity of light detected at the light source sensor 117 is at a maximum, the light or lights corresponding to maximum light detection is illuminated, for example. In certain embodiments, the receiver 112 may provide a quantitative value of the intensity of the signal. For example, the receiver 112 may be connected to a monitor or a display module that produces a numerical or quantitative value indicating the intensity of light perceived by the light source sensor 117. In certain embodiments, the receiver may generate both an audible and a visual indicator signal.

The operator may therefore determine an accurate position of a feeding tube inside of a patient by locating the light source position that yields the highest intensity indicator signal, as produced by the receiver. In certain embodiments, a predetermined intensity value may confirm the presence of the light source sensor 117 and thus the feeding tube 126. For example, where the receiver 112 has a light display ranging in intensity level from zero to ten, wherein a value of zero indicates no perceived light, and 10 indicates a maximal or near maximal amount of perceived light, it may be predetermined that an intensity level of 7 or greater is sufficient to confirm the location of the tube 126.

In addition to the systems described, methods for locating the position of a feeding tube inside of a patient's body are also provided. In certain embodiments, the method comprises the following steps:
1) Providing a feeding tube having a distal end with a proximity target. The proximity target may be a metal band as disclosed above.
2) Inserting the feeding tube into the patient. In certain embodiments, the tube may be inserted through the nose or the mouth. In certain embodiments the tube may pass through the esophagus into the stomach. In certain embodiments, the tube may pass beyond the stomach and into the small intestine.
3) Placing a detector above the patient. For example, where a feeding tube having a metal band is placed inside of a patient's stomach, a metal detector may be placed above the patient's stomach area.
4) Generating a signal, indicating the detection of the proximity target. For example, where the detector is a metal detector, the detector may produce a sound indicating the detected presence of the target. In certain embodiments, the detector may produce a visual signal in addition to, or instead of, an audible signal indicating the detected presence of the target.

Other embodiments provide methods for locating the position of a feeding tube inside of a patient, where the sensor is a light source sensor in the feeding tube inserted into the patient, detecting a light source external to the body of the patient. The method comprises the following steps:

1) Providing a feeding tube having a light source sensor at the distal end.

2) In certain embodiments, the light source sensor of the feeding tube is connected to a receiver. The receiver or sensor may be located exterior to the patient's body.

3) In certain embodiments, the feeding tube having the light source sensor is then inserted inside of a patient. For example, the feeding tube may pass through a patient's nose or mouth, down the esophagus into the stomach of the patient, or beyond the stomach and into the small intestine. In certain embodiments, the feeding tube may be inserted into the patient before the light source sensor is connected to the receiver. For example, the operator or technician may prefer not to have the sensor connected to a receiver while inserting into the patient in order to have a greater available range of movement of the tube.

4) Next, a non-radiographic light is shone externally above the patient. For example, a user may shine an infrared light from a light source above an area of the patient's body approximately where the feeding tube is expected to be located.

5) Next, an indication signal is generated reflecting the detection of light by the light source sensor. In certain embodiments, the signal generated may be based upon the intensity of the light perceived. For example, the receiver may generate a sound that increases in pitch or volume as the intensity of the light detection signal transmitted by the light source sensor increases. Alternatively, the receiver may produce an intermittent sound that increases in frequency with an increase in intensity based on the intensity of the light perceived. In certain embodiments the indication signal may be visual. For example, in certain embodiments a light source may be provided that varies in color, blinking frequency, or amount of lights powered based on the intensity of the light detection signal received by the receiver or sensor.

6) In certain embodiments, the position of the feeding tube within the patient's body is located by moving the light source over the body to the point where the indication signal is of highest intensity. For example, an operator may move the light source about a patient's stomach and note the location that causes the receiver to generate the signal indicating the highest perceived intensity of light. In certain embodiments, the light source may be moved to a location where the indicator signal reaches or exceeds an intensity level that is predetermined to confirm the presence of the light source sensor, and thus the feeding tube.

The above mentioned steps are not limited to be performed in the order in which they are listed. For example, the feeding tube may be inserted into a patient first, after which the light source sensor may be connected to a receiver.

The different embodiments of the presently described methods and systems offer techniques that provide several advantages over conventional methods of detecting the position of a feeding tube inside of a patient. The presently described techniques are more accurate than those provided by use of pH, gastric enzymes, air or CO₂ detectors. Furthermore, the techniques allow for the location of the tube to be easily confirmed at any time during use of the feeding tube, not just during insertion. Additionally, the present techniques do not rely on radiographic methods to detect the position of the feeding tube. Furthermore, the detector is simple and easy to use by either medical professionals or medically trained friends and family of the patient, and thus the techniques may be used either at a medical facility or a home care setting.

Variations and modifications of the foregoing are within the scope of the present technology. It is understood that the technology disclosed and defined herein extends to all alternative combinations of two or more of the individual features mentioned or evident from the text and/or drawings. All of these different combinations constitute various alternative aspects of the present technology. The embodiments described herein explain the best modes known for practicing the technology and will enable others skilled in the art to utilize the technology. The claims are to be construed to include alternative embodiments to the extent permitted by the prior art.

1. A system for detecting the location of a feeding tube in a patient's body, said system comprising:
a feeding tube having a light source sensor;
a receiver connected to said light source sensor; and
a light source generating non-radiographic light;
wherein said feeding tube is inserted into a patient and said receiver generates an indication signal when said light source sensor detects non-radiographic light from said light source.

3. The system of claim 1 wherein said light source sensor is a passive infrared sensor, and said non-radiographic light is infrared light.

4. The system of claim 1, wherein said light source sensor is at least one fiber-optic filament.

5. The system of claim 1, wherein said indication signal is audible.

6. The system of claim 1, wherein said indication signal is produced by at least one display light on said receiver.

7. The system of claim 1, wherein said light source is hand held.

8. The system of claim 7, wherein said light source is operable with either the use of a right or a left hand.

9. The system of claim 1, wherein said light source sensor is removably attached to said receiver via a joint.

10. The system of claim 1, wherein said feeding tube is located in at least one of the stomach and the small intestine of the patient.

11. The system of claim 1, wherein said feeding tube has a radiographic pigment for X-ray detection.

12. The system of claim 1, wherein said feeding tube comprises measurement markings along the length of the tube.

13. The system of claim 1, wherein said feeding tube comprises a dual feeding administration ports.

14. The system of claim 1, wherein said system is portable.

15. The system of claim 1, wherein said light source sensor detects the intensity of light from said light source and said indication signal is based on the intensity of the light detected by said light source sensor.

16. The system of claim 15, wherein said indication signal is audible and varies in at least one of pitch and volume based upon the intensity of said light detected from said light source.

17. The system of claim 15 wherein said indication signal is generated by a series of display lights on said receiver, said display lights illuminating based on the intensity of said light detected from said light source sensor.

18. The system of claim 15, further comprising a display unit, wherein said receiver generates a quantitative value of the intensity of said light detection and said value is displayed on said display unit.

19. A method for detecting the location of a feeding tube inside of a patient comprising the following steps:
inserting a feeding tube having a light source sensor at the distal end of said feeding tube into a patient;
generating a non-radiographic light over the patient;
detecting the presence of light at the distal end of said light source sensor; and
generating an indicator signal based on the detected presence of said non-radiographic light detected at said light source sensor.

20. The method of claim 19, wherein said indicator signal is generated by a receiver connected to said light source sensor.

21. The method of claim 19, further comprising detecting the intensity of light at said light source sensor and generating an indicator signal that is based on said intensity of light.

22. The method of claim 21, wherein said indicator signal is an audible sound that varies in at least one of pitch and volume based on said intensity of light detected at said light source sensor.

23. The method of claim 21, wherein said indicator signal is generated on a visual display, said display comprising a series of display lights that illuminate based on said intensity of light detected at said light source sensor.

24. The method of claim 21, wherein said non-radiographic light is generated by a light source and said light source is moved externally over the patient to the point where said indicator signal indicates that the intensity of light detected at said light source sensor is at or above a predetermined value.

25. A system for detecting the location of a feeding tube in a patient comprising:
a feeding tube having a proximity sensor target; and
a detector having a proximity sensor, wherein said feeding tube is inserted into the patient and said detector is externally moved about the patient's body such that, when said proximity sensor detects said proximity sensor target of said feeding tube, said detector produces an indication signal.
